(19) United States
(12) Patent Application Publication
(10) Pub. No.: US 2025/0197329 A1
(43) Pub. Date: Jun. 19, 2025

(54) 1,3-BUTANEDIOL

(71) Applicant: Resonac Corporation, Tokyo (JP)

(72) Inventors: Takuya MORIWAKI, Oita-shi (JP); Kazuya KIMURA, Oita-shi (JP)

(73) Assignee: Resonac Corporation, Tokyo (JP)

(21) Appl. No.: 19/063,961

(22) Filed: Feb. 26, 2025

Related U.S. Application Data
(63) Continuation of application No. 17/438,117, filed on Sep. 10, 2021.

(30) Foreign Application Priority Data
Dec. 24, 2019 (JP) 2019-232310

(51) Int. Cl. C07C 31/20; A61K 8/34; A61Q 19/00

(52) U.S. Cl. CPC C07C 31/207; A61K 8/345; A61Q 19/00; A61K 2800/10

(57) ABSTRACT

The purpose of the present invention is to stably provide 1,3-butanediol which is less odorous and is suitable for use in cosmetics. The 1,3-butanediol has a content of odorous substance A, which is represented by chemical formula (A), of 2-10 wtppm and a content of odorous substance B, which is represented by chemical formula (B), of 4-25 wtppm.

### CROSS REFERENCE TO RELATED APPLICATIONS

This application is a continuation of application Ser. No. 17/438,117, filed Sep. 10, 2021, which is a National Stage of International Application No. PCT/JP2020/046088 filed Dec. 10, 2020, which claims priority based on Japanese Patent Application No. 2019-232310 filed Dec. 24, 2019. The disclosure of application Ser. No. 17/438,117 is incorporated herein by reference in its entirety.

### FIELD

The present invention relates to low odor 1,3-butanediol suitable for cosmetic applications.

### BACKGROUND

1,3-butanediol is a viscous, colourless, transparent, odorless, water-soluble liquid with a boiling point of 207° C. and is used as a raw material for various derivatives. For example, long-chain carboxylic acids and esters formed from 1,3-butanediol have been utilized as plasticizers. In addition, 1,3-butanediol is also used as a cosmetic raw material because of its low biotoxicity and its stability. Since 1,3-butanediol has characteristics, such as a moisturizing effect, an antibacterial property, and little stickiness, it is used as a cosmetic raw material for a wide range of products, such as shampoos, emulsions, and moisturizers. Among them, in the case of a cosmetic application, such as a moisturizer, 1,3-butanediol having little odor is required. Although 1,3-butanediol itself is almost odorless, there may be some cases that odors have been generated due to by-products and impurities generated during the manufacturing process.

One of the main methods for the preparation of 1,3-butanediol includes the condensation of acetaldehyde to give acetaldol (3-hydroxybutanal), which is then hydrogenated. However, acetaldol itself is unstable and difficult to handle as a single substance.

Therefore, in practice, acetaldehyde is condensed in the presence of a basic catalyst to obtain aldoxane (a common name of 2,6-dimethyl-1,3-dioxane-4-ol), and acetaldehyde generated by heating and decomposing the aldoxane is distilled off to obtain paraldol (a common name of 4-hydroxy-α,6-dimethyl-1,3-dioxane-2-ethanol) which is a dimer of acetaldehyde (Patent Literature 1).

Then, this paraldol is used as a raw material for the hydrogenation reaction to produce 1,3-butanediol. Aldoxane may be used as a raw material for the hydrogenation reaction, and in this case, although ethanol is by-produced, 1,3-butanediol can be produced.

As a method of obtaining 1,3-butanediol with little odor, for example, JPH07-258129A (Patent Literature 2) describes a method, in which a compound, such as caustic soda, is added in the distillation for removing high boiling point compounds. In addition, WO2000/07969 (Patent Literature 3) describes a method, in which an alkali metal base is added to a crude 1,3-butanediol from which high boiling point compounds have been removed, followed by the heat treatment, and then 1,3-butanediol is distilled off to separate the alkali metal compound and high boiling point compounds as residue, followed by the distillation for removing low boiling point compounds from the 1,3-butanediol fraction. However, 1,3-butanediol obtained from any of the above methods still has an odor, and the odor substance has not been clarified. Therefore, it has not been possible to quantify the degree of purity of the raw material and the degree of purification required. JP2003-096006A (Patent Literature 4) describes 1,3-butanediol having little odor, but a specific odor substance is not identified. JP5024952B (Patent Literature 5) describes a dioxane type compound as an odor substance in an alkanediol composition having 4 or more carbon atoms. However, only a general formula of a dialkyl dioxane is described, and there is no description of an odor substance to be specifically reduced, and a true odor substance was unknown.

In order to obtain 1,3-butanediol having little odor, it was necessary to design a facility having an excessive purification capacity, and it was very difficult to produce 1,3-butanediol having little odor stably and economically.

### CITATION LIST

### Patent Literature

[Patent Literature 1] JPS62-212384A

[Patent Literature 2] JPH07-258129A

[Patent Literature 3] WO2000/07969

[Patent Literature 4] JP2003-096006A

[Patent Literature 5] JP5024952B

### SUMMARY

### Technical Problem

It is an object of the present invention to stably provide 1,3-butanediol with little odor by quantifying and managing the odor substances in 1,3-butanediol.

### Solution to Problem

As a result of extensive studies on the above problems, the present inventors have found that an odor in 1,3-butanediol is derived from a plurality of odor substances, and have identified them to complete the present invention.

The present invention includes the following [1] to [3].

[1]

A 1,3-butanediol, wherein an odor substance A represented by the chemical formula (A) is 2 wtppm or more and 10 wtppm or less, and an odor substance B represented by the chemical formula (B) is 4 wtppm or more and 25 wtppm or less.

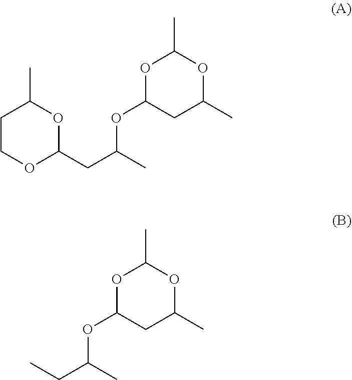

[2]

The 1,3-butanediol according to [1], which is used as a cosmetic raw material.

[3]

A cosmetic comprising the 1,3-butanediol according to [1] or [2].

### Advantageous Effects of Invention

According to the present invention, 1,3-butanediol having little odor is stably and economically provided.

### BRIEF DESCRIPTION OF THE DRAWINGS

FIG. 1 is a chromatogram of the 1,3-butanediols of Example 1, Comparative Example 1, and Comparative Example 4.

FIG. 2 is a chromatogram enlarged view in the vicinity of the peaks of the odor substance A in the chromatogram of the 1,3-butanediols of Example 1, Comparative Example 1, and Comparative Example 4.

FIG. 3 is a chromatogram enlarged view in the vicinity of the peaks of the odor substance B in the chromatogram of the 1,3-butanediols of Example 1, Comparative Example 1, and Comparative Example 4.

FIG. 4 is a graph showing the contents of the odor substance A and the odor substance B of the 1,3-butanediols of Examples and Comparative Examples.

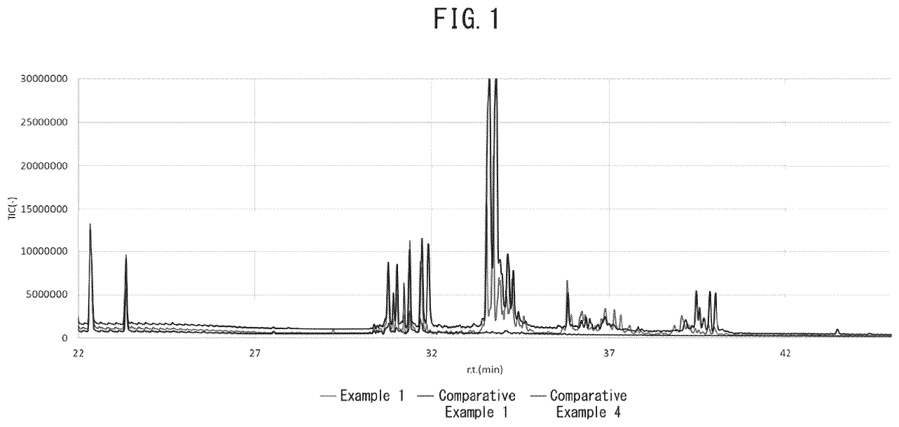

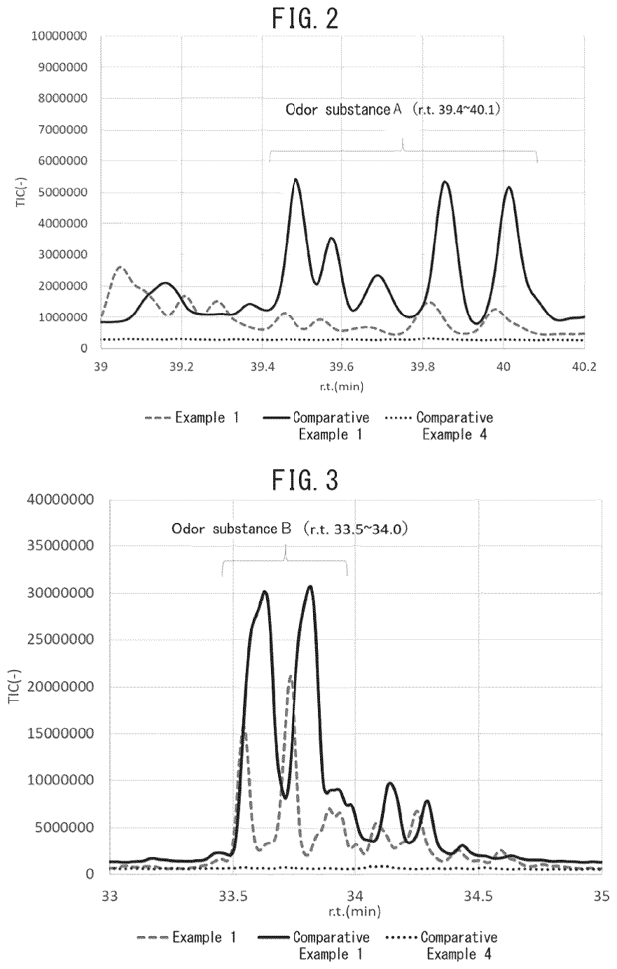

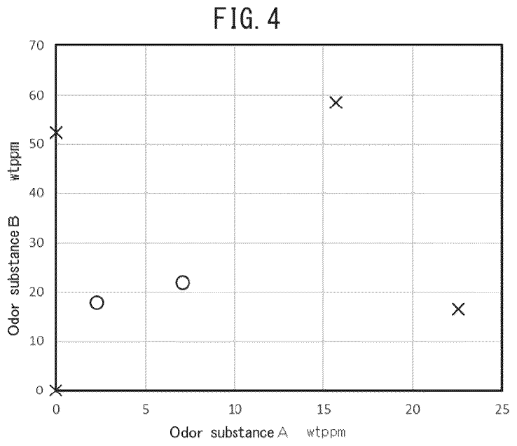

### DESCRIPTION OF EMBODIMENTS

Hereinafter, preferred embodiments of the present invention will be described, but the present invention is not limited to these embodiments only.

A 1,3-butanediol according to an embodiment can be obtained by purifying a crude 1,3-butanediol. The method for producing the crude 1,3-butanediol is not particularly limited, and can be produced by, for example, a known method (see JPH03-80139B, JPH07-258129A, and the like).

Specifically, as shown in the following reaction formula, acetaldehyde can be used as a starting material, and paraldol can be hydrogenated to obtain 1,3-butanediol.

### 1. Condensation Step

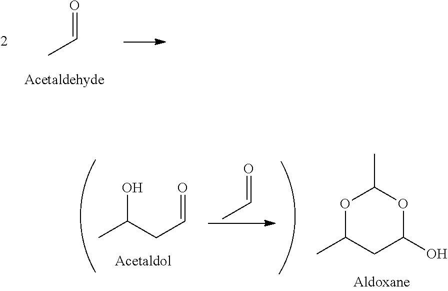

### 2. Thermal Decomposition Step

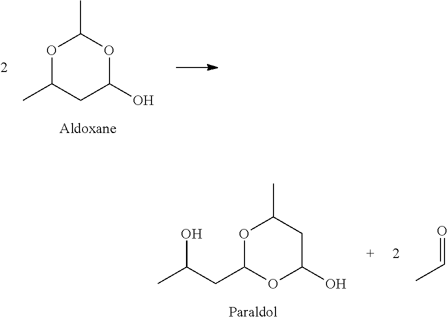

### 3. Hydrogenation Step

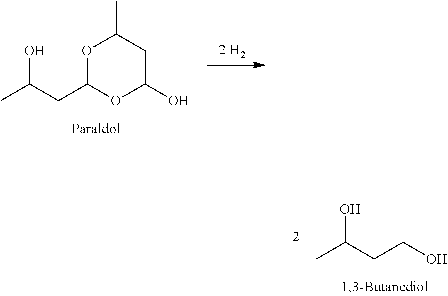

### 1. Condensation Step

The condensation step is a step of obtaining acetaldol, or further aldoxane, from acetaldehyde. The acetaldols are the raw materials for the hydrogenation reaction, and the method for producing the same is not particularly limited. For example, the acetaldols are prepared by the following method.

By reacting acetaldehyde with a catalytic amount of base, two molecules of acetaldehyde react to give one molecule of acetaldol. As the base, for example, sodium hydroxide or potassium hydroxide can be used. Since the generated acetaldol is unstable, one molecule of acetaldol rapidly reacts with one molecule of acetaldehyde to yield one molecule of aldoxane. In the present disclosure, such a reaction of obtaining acetaldol from acetaldehyde and further obtaining aldoxane is referred to as a condensation reaction, and a step of carrying out a condensation reaction is referred to as a condensation step.

Since the condensation reaction is an equilibrium reaction, when the reaction mixture approaches the equilibrium composition, the progress of the reaction becomes slow. When the base is present in this state, a high boiling component, such as a trimer, which has undergone further condensation, is generated from acetaldol, or crotonaldehyde is generated by the dehydration of acetaldol. Therefore, if necessary, an acid is added to neutralize the base in order to stop the reaction. As the acid, for example, an organic acid, such as acetic acid, can be used.

The condensation reaction can be carried out in a liquid phase at a temperature from 20 to 50° C., a pressure from 0.1 to 0.2 MPaG (gauged pressure), and a reaction time from 2 to 20minutes. The reaction atmosphere is preferably under an inert gas, such as nitrogen gas or argon. There is no limitation on the reactor used in the condensation reaction, and for example, a tank type reactor can be used.

### 2. Thermal Decomposition Step

Even though hydrogenation of the aldoxane obtained in the condensation step can also give 1,3-butanediol, one molecule of ethanol is produced from one molecule of aldoxane together with one molecule of 1,3-butanediol. Therefore, when co-production of ethanol is not preferable, if necessary, aldoxane is converted into paraldol by a thermal decomposition reaction of aldoxane, and the obtained paraldol is hydrogenated. Thus, 1,3-butanediol can be obtained without causing by-product of ethanol.

When aldoxane is heated, one molecule of aldoxane decomposes into one molecule of acetaldol and one molecule of acetaldehyde by the equilibration reaction. Then, under certain temperature and pressure conditions, acetaldehyde vaporizes and is removed from the system. At this time, the remaining two molecules of acetaldehyde associate to generate one molecule of paraldol. The by-produced acetaldehyde can be reused as a starting material. In this disclosure, such a reaction of obtaining paraldol and acetaldehyde from aldoxane is referred to as a thermal decomposition reaction, and a step of carrying out a thermal decomposition reaction is referred to as a thermal decomposition step.

Hydrogenation of one molecule of paraldol gives two molecules of 1,3-butanediol. Therefore, when the hydrogenation reaction is carried out after aldoxane is completely converted into paraldol by advancing the thermal decomposition reaction, no ethanol is produced at all as the by-product. However, in the process of converting aldoxane into paraldol, generation of crotonaldehyde by the dehydration of acetaldol, and generation of the high boiling components by the polymerization of acetaldol, crotonaldehyde, and the like, occur. For this reason, in practice, the thermal decomposition reaction of aldoxane is stopped at an appropriate conversion rate to obtain a mixture of aldoxane and paraldol as a thermal decomposition reaction solution.

The thermal decomposition reaction can be carried out in a liquid phase at a temperature from 60 to 80° C., a pressure from 0.01 to 0.1 MPaG, and a reaction time from 20 to 90 minutes. The reaction atmosphere is preferably under an inert gas, such as nitrogen gas or argon.

In the hydrogenation step serving as the next step, after separating paraldol and aldoxane in the thermal decomposition reaction solution, only paraldol may be used as a raw material for the hydrogenation reaction. Alternatively, since the separation of paraldol and aldoxane is difficult by a general separation method, such as distillation, they may be used as a raw material for the hydrogenation reaction as a mixture without separation. The raw material of the hydrogenation reaction may contain not only crotonaldehyde or a high boiling component generated in the thermal decomposition step, but also a salt generated by the neutralization of the base used in the condensation step.

### 3. Hydrogenation Step

Paraldol obtained in the thermal decomposition step is hydrogenated by contacting it with a hydrogenation catalyst in the presence of hydrogen gas (H₂) and converted to 1,3-butanediol. It is also possible that aldoxane, which is a raw material of the thermal decomposition step and which has not been reacted, is simultaneously hydrogenated to obtain 1,3-butanediol. In the present disclosure, a step of carrying out a hydrogenation reaction is referred to as a hydrogenation step.

The temperature at which the hydrogenation reaction is carried out may be from 50 to 150° C., preferably from 70 to 130° C. By setting the temperature to 50° C. or higher, the hydrogenation reaction can reliably proceed, and by setting the temperature to 150° C. or lower, side reactions, such as a hydrogenolysis reaction, can be suppressed and the yield of the target product, 1,3-butanediol, can be increased.

The pressure at which the hydrogenation reaction is carried out may be from 5 to 15MPaG, preferably from 7 to 12 MPaG. By setting the pressure to 5 MPaG or more, the hydrogenation reaction can be promoted, and by setting the pressure to 15 MPa or less, the cost for boosting the pressure of hydrogen and the facility cost can be reduced.

Any hydrogenation catalyst can be used, and a nickel-based catalyst is generally effective. In particular, stabilized nickel in which nickel is supported on a carrier, such as alumina or silica, and sponge nickel in which aluminum is eluted from an alloy of nickel and aluminum are effective.

There is no particular limitation on the reactor for carrying out the hydrogenation reaction, and for example, a tank type reactor can be used.

In addition to 1,3-butanediol, various low boiling components are contained in the reaction solution obtained in the hydrogenation step. Examples of the low boiling component include ethanol generated mainly by hydrogenating aldoxane, and 1-butanol, 2-butanol, and 2-propanol produced as by-products during the hydrogenation of acetaldol. The low boiling component may include water brought from the condensation step or the thermal decomposition step.

These low boiling components can be removed by a separation operation, such as distillation, after the hydrogenation reaction. The low boiling component can be discarded or effectively utilized as other chemical raw materials after separating useful compounds.

The crude 1,3-butanediol from which the low boiling component has been removed is purified by one or more separation operations to a practical purity, thereby obtaining a crude 1,3-butanediol, which will be a product other than for cosmetic use.

There is no particular limitation on the method for purifying the crude 1,3-butanediol to obtain 1,3-butanediol which can be used as a cosmetic raw material. For example, a known method of removing ethanol, which is a by-product, by distillation from a reaction product obtained by hydrogen reduction of acetaldol (see JPH03-80139B and JPH07-258129A and the like), a method of carrying out additional one or more known purification steps on the fraction after removing ethanol, and the like may be mentioned, and a known purification step may be carried out repeatedly. As a known purification method, for example, a distillation for removing a high boiling component, a distillation in which water is introduced from the top of a column to extract 1,3-butanediol from the bottom of the column, or a step in which crude 1,3-butanediol is mixed with water and water is evaporated to obtain 1,3-butanediol, a step of extracting impurities with an organic solvent (e.g., pentane, hexane, toluene, and the like), a step of adding an alkali metal compound (e.g., sodium hydroxide, potassium hydroxide, and the like) and heat-treating the mixture, a step of removing impurities using an adsorbent, such as activated carbon, and the like, may be mentioned.

The content of the odor substance A represented by the chemical formula (A) contained in the 1,3-butanediol of the present embodiment is not less than 1 wtppm and not more than 10 wtppm, and the content of the odor substance B represented by the chemical formula (B) is not less than 4 wtppm and not more than 25 wtppm.

Preferably, the content of the odor substance A is not less than 2 wtppm and not more than 8 wtppm, and the content of the odor substance B is not less than 8 wtppm and not more than 20 wtppm.

More preferably, the content of the odor substance A is not less than 2 wtppm and not more than 8 wtppm, and the content of the odor substance B is not less than 12 wtppm and more and not more than 18 wtppm.

The content of the odor substance A is more preferably not more than 8 wtppm, and still more preferably not more than 5 wtppm.

The content of the odor substance A is more preferably not less than 2 wtppm.

The content of the odor substance B is more preferably not more than 20 wtppm, and still more preferably not more than 18 wtppm.

The content of the odor substance B is more preferably not less than 8 wtppm, and still more preferably not less than 12 wtppm.

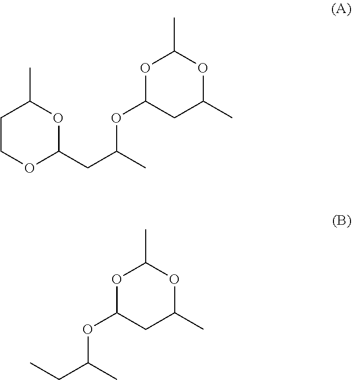

The 1,3-butanediol of the present embodiment has little odor and is suitable as a raw material for cosmetic use, such as a moisturizer.

In addition, the 1,3-butanediol of the present embodiment does not require an excessive purification step for removing odor substances in its production, and can be produced stably and at low cost.

### EXAMPLES

Hereinafter, embodiments of the present invention will be described specifically, but the present invention is not limited to the examples.

### 1. Odor Intensity:

As evaluation criteria, 1,3-butanediol, which does not provide an odor, was scored 0, and 1,3-butanediol, which was almost odorless, was scored 1, and that with a slight odor was scored 2, and scores were given in the relative evaluation. The evaluation samples were placed in stoppered wide-mouthed reagent bottles, sealed tightly, and allowed to stand at room temperature, and then quickly smelled in the air, compared, and scored. The evaluation was carried out by 3 adults, and the average value of the scores was adopted.

### 2. GC-MS Analyses:

Sample preparation method: After adding 240 g of distilled water to 60 g of 1,3-butanediol of the sample, 90 g of cyclohexane was added and shaken to extract the organic substance into cyclohexane. The aqueous phase and cyclohexane phase were separated, and approximately 90 g of the cyclohexane phase was concentrated to 0.2 g in an evaporator under a reduced pressure of 100 to 150 torr at 30 to 40° C. to prepare a sample for GC-MS analysis.
GC-Analyzer: Agilent 7890B (manufactured by Agilent Technologies, Inc.)
Mass spectrometer: Quadrupole type MS JMS-T100GCV (manufactured by JEOL Ltd.)
Ionization method: EI+, FI+.
Analytical columns: DB-1MS (60 m, 0.32 mm, 0.25 μm) (manufactured by Agilent Technologies, Inc.)
Column-heating condition: 50° C. (2 minutes)→5° C./minute→250° C. (10 minutes)
Carrier gas: He
Split ratio: 10:1
Sample injection volume: 2 μL
Internal reference material: Xylene

Peaks derived from the odor substance A are shown as a group of peaks (four peaks due to the presence of optical isomers) with retention time (r.t) of 39.4 to 40.1 minutes. The content of the odor substance A was quantified using the integrated area, and a calibration curve prepared from a standard material of the odor substance A and the internal reference material.

Peaks derived from the odor substance B are shown as a group of peaks (two peaks due to the presence of optical isomers) with retention time (r.t) of 33.5 to 34.0 minutes. Assuming that the factors of the odor substance A and the odor substance B were equal, the content of the odor substance B was quantified using the integrated area, and a calibration curve prepared from a standard material of the odor substance A and the internal reference material.

The standard material of the odor substance A was synthesized by heating 1,3-butanediol and acetaldol at about 60° C. in the presence of an acid catalyst (p-toluenesulfonic acid) to obtain an acetal compound, mixing the acetal compound and aldoxane, heating the mixture at about 60° C. in the presence of an acid catalyst (p-toluenesulfonic acid), and then separating the mixture on an open column.

### Comparative Example 1

10 g of paraldol as acetaldols, 40 g of ethanol, and 1 g of a sponge nickel catalyst (R-201, manufactured by Nikko Rica Corporation) were added into a 120 mL autoclave made of SUS316L. The autoclave was pressurized to 8 MPaG with hydrogen gas and stir was initiated. 1,3-Butanediol was obtained by raising the temperature at a rate of 1° C./min from 30° C. to 120° C. and cooling the autoclave immediately at the moment when the temperature reached 120° C. to stop the reaction. During the reaction, hydrogen gas was supplied to a pressure of 8 MPaG each time the pressure dropped to 7 MPaG. After filtering off the catalyst, the reaction solution was distilled using a distillation column having 10 or more theoretical plates under a reduced pressure of 100 torr or less and a temperature of 150° C. or less, whereby ethanol was separated as a low boiling component, and then crude 1,3-butanediol was obtained. The results of the hydrogenation reaction were 97.5% of paraldol conversion and 96.5% of 1,3-butanediol selectivity. The concentrations of the odor substance A and the odor substance B of the crude 1,3-butanediol were 16 wtppm and 58 wtppm, respectively. The odor intensity was 2.

### Example 1

100 parts by mass of the crude 1,3-butanediol obtained in Comparative Example 1 and 100 parts by mass of water were mixed to obtain a solution, which was distilled (using a distillation column having 5 or more theoretical plates under a reduced pressure of 100 torr or less and a temperature of 150° C. or less) to remove water as a low boiling component to obtain 1,3-butanediol. The concentrations of the odor substance A and the odor substance B of the 1,3-butanediol were 2 wtppm and 18 wtppm, respectively, and the odor intensity was 0.

### Example 2

The crude 1,3-butanediol obtained in Comparative Example 1 was distilled (using a distillation column having 10 or more theoretical plates under a reduced pressure of 100 torr or less and a temperature of 150° C. or less) to remove high boiling components to obtain 1,3-butanediol. The concentrations of the odor substance A and the odor substance B of the 1,3-butanediol were 7 wtppm and 22 wtppm, respectively, and the odor intensity was 0.

### Comparative Example 2

A commercially available 1,3-butanediol (Commercial Product 1) was evaluated.

The concentrations of the odor substance A and the odor substance B of the Commercial Product 1 were 0 wtppm and 52 wtppm, respectively, and the odor intensity was 2.

### Comparative Example 3

A commercially available 1,3-butanediol (Commercial Product 2) was evaluated.

The concentrations of the odor substance A and the odor substance B of the Commercial Product 2 were 23 wtppm and 17 wtppm, respectively, and the odor intensity was 6.

### Comparative Example 4

20 parts by mass of the crude 1,3-butanediol obtained in Comparative Example 1, 80 parts by mass of water, and 30 parts by mass of cyclohexanone were mixed, and 1,3-butanediol was dissolved in the aqueous phase. After extracting the impurities into the cyclohexanone phase, the aqueous phase and the cyclohexanone phase were separated. The aqueous phase was distilled to remove water as a low boiling component using a distillation column having 5 or more theoretical plates under a reduced pressure of 100 torr or less and a temperature of 150° C. or less to obtain 1,3-butanediol from the bottom of the column. The concentrations of the odor substance A and the odor substance B of the 1,3-butanediol were 0 wtppm and 0 wtppm, respectively, and the odor intensity was 0.

Table 1 shows the evaluation results. From this result, it can be understood that the odor does not disappear unless both of the odor substance A and the odor substance B are within a specific content range.

Further, when the degree of purification is increased, it is possible to almost remove the odor substance A and the odor substance B as shown in Comparative Example 4. However, it is disadvantageous for industrial production, since the production cost increases.

TABLE 1

| | Odor | Odor | |
|---|---|---|---|
| | substance | substance | Odor |
| | A [wtppm] | B [wtppm] | intensity |
| Example 1 | 7 | 18 | 0 |
| Example 2 | 7 | 22 | 0 |
| Comparative | 16 | 58 | 2 |
| Example 1 | | | |
| Comparative | 0 | 52 | 2 |
| Example 2 | | | |
| Comparative | 23 | 17 | 6 |
| Example 3 | | | |
| Comparative | 0 | 0 | 0 |
| Example 4 | | | |

### INDUSTRIAL APPLICABILITY

The present invention provides 1,3-butanediol, which is odorless and of extremely high quality, and can be produced economically.

What is claimed is:

1. A method for producing a 1,3-butanediol composition, the method comprising a purifi cation step of a crude 1,3-butanediol obtained by hydrogenation of paraldol, wherein the purification step comprises a water-added distillation step in which after a step of separating ethanol as a low boiling component from the crude 1,3-butanediol, the obtained crude 1,3-butanediol and water are mixed to obtain a solution, and the obtained sol ution is distilled to remove water, and the 1,3-butanediol composition comprises an odor substance A represented by the che mical formula (A) in an amount of 1 wtppm or more and 10 wtppm or less, and an odor s ubstance B represented by the chemical formula (B) in an amount of 4 wtppm or more and 25 wtppm or less, based on the weight of the 1,3-butanediol composition:

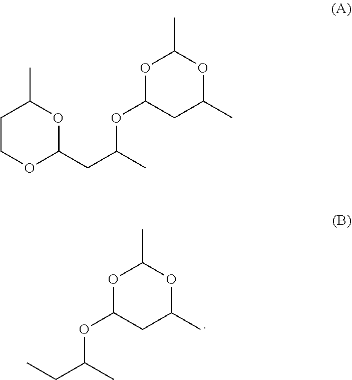

2. The method according to claim 1, wherein the water-added distillation step is performed at the end of the purification step.
